(19) United States
(12) Patent Application Publication
(10) Pub. No.: US 2004/0138651 A1
(43) Pub. Date: Jul. 15, 2004

(54) Pericardial maze procedure

(76) Inventors: James R. Grabek, Minneapolis, MN (US); Michael Hoey, Shoreview, MN (US)

Correspondence Address: Beck & Tysver, P.L.L.C., Suite 100, 2900 Thomas Avenue S., Minneapolis, MN, 55416, US

(21) Appl. No.: 10/691,844

(22) Filed: Oct. 23, 2003

Related U.S. Application Data
(63) Continuation-in-part of application No. 09/565,059, filed on May 3, 2000.
(60) Provisional application No. 60/421,548, filed on Oct. 25, 2002.

(51) Int. Cl. A61B 18/18

(52) U.S. Cl. 606/32; 606/41

(57) ABSTRACT

A device for performing the maze procedure through the pericardial space, which includes a very flexible catheter for laying down lesions in conformity with the atrium of the heart.

### CROSS REFERENCE TO RELATED APPLICATIONS

The present application is a Continuation-In-Part of U.S. patent application Ser. No. 09/565,059 filed May 3, 2000 which is incorporated in its entirety herein. The present application claims the benefit of provisional application 60/421,548 filed Oct. 25, 2002, which is incorporated in its entirety herein.

### BACKGROUND OF THE INVENTION

Control of arthymias, especially artrial arthymias is typical to achieve through the use of drugs or implantable stimulators. To address the shortcoming of these prior procedures, physicians have developed a surgical intervention called the maze technique. In a maze procedure the heart is surgically cut along lines in the atrium which form scar tissue. The presence of the scar tissue interrupts the arthymia circuits within the tissue. Surgical maze, although effective, is extremely invasive requiring open chest and open heart surgery.

More recently the maze procedure has been accomplished through the use of a radio frequency (RF) ablation catheter inside the atrium. In this technique a conventional electrophysiology (EP) or ablation catheter is used to lay down a sequence of “burns” which destroy tissue and interrupt the arthymia circuits in the heart. This technique is substantially less invasive than surgical maze, but has numerous shortcomings as well. It is difficult to maintain adequate coupling between the ablation electrode and the cardiac surface within the blood pool, and this results in inadequate bum depth and the accumulation of coagulum on the lead. The inability to visualize and return to locations within the atrium also make it difficult to provide the line of scar tissue which is the hallmark of a maze procedure.

### SUMMARY OF THE PRESENT INVENTION

In contrast to prior art techniques Applicant proposes the use of novel devices and methods to achieve a maze procedure from within the pericardial space. Access to the pericardial space may be achieved using a conventional needle technique or the PerDUCER device sold by Comedicus of Minneapolis Minn. or a grasping pericardial device may be used. Other approaches may be used as well, including that taught by U.S. Patent Appln No. US 2002/0058925 Pub. May 16, 2002. The choice of pericardial access is not restrictive in practicing the invention.

Once a guide wire is introduced into the pericardial space the specialized ablation lead may be introduced. Guide wires are not necessary for placement of the device, but they are useful in this context. In addition to the ablation lead a laparoscope or other optical visualization device may be introduced into the space. A laparoscope or other camera-type device may be introduced as well.

There are several embodiments of the specialized ablation lead shown. In one embodiment a fixation element is placed at the distal tip of the device so that it may be temporarily anchored to cardiac tissue. Both mechanic fixation and suction attachment are shown. One or more fixation sites may be provided on the lead. Multiple electrode sites or a single long electrode site are provided on the body of the lead to lay down the linear lesion upon the application of RF energy.

In an alternate embodiment a stearable device having a stylet or other stiffening member is introduced to help guide the electrode structures to their intended treatment location. Once the lead has been positioned the stylet is removed to permit the multi-link electrodes to conform to the tissue surface, both statically and dynamically as the heart beats. In this fashion better electrical contact is provided with the heart permitting good electrical contact.

In the anchored embodiments the burn is laid down with the lead or catheter in a static position both helical fixation tips and vacuum concepts are taught in the application. In the stylet stiffened version a “burn and drag” linear ablation technique may be used where the coil is exposed to the tissue and dragged along its surface while the RF generator is operating.

Although the techniques and devices taught herein are particularly well suited for atrial maze lesions it must be understood that this use is only representative and other uses and in particular ventricular uses are contemplated.

### BRIEF DESCRIPTION OF THE DRAWINGS

In the figures identical reference numerals indicate similar or identical structure wherein:

FIG. 1 is a schematic view of the structure and use of the device;

FIG. 2 is divided into panels 2a, 2b, 2c showing a sequence of method steps;

FIG. 3 is a schematic view of the structure and use of the device;

FIG. 4 is a schematic view of the structure and use of the device;

FIG. 5 is a schematic view of the structure and use of the device; and,

FIG. 6 is a schematic view of the structure and use of the device.

### DETAILED DESCRIPTION OF DRAWINGS

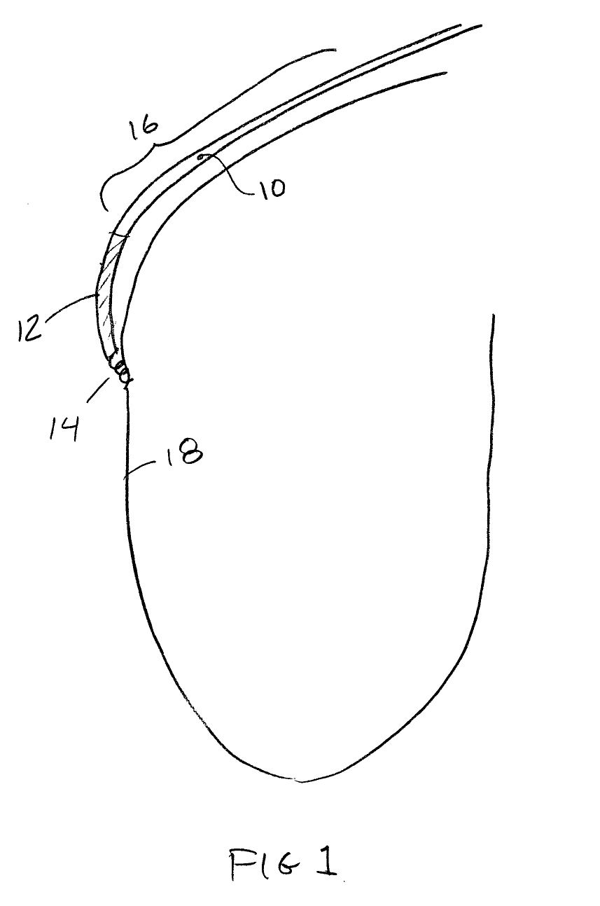

In FIG. 1 the ablation catheter 16 has entered the pericardium and is between the pericardial sack and the epicardial surface. A portion of the catheter 16 is insulated as indicated by surface 10. A distal portion of the catheter 16 is an exposed electrode 12. This distal surface is coupled to helical screw 14 which can be rotated by the catheter 16 to temporarily fix the catheter to the surface of the heart. Once anchored by the tip 14 the catheter may be manipulated to lay down a series of linear lesions along the heart surface. The energy for the ablation will be supplied from an RF generator in the conventional fashion.

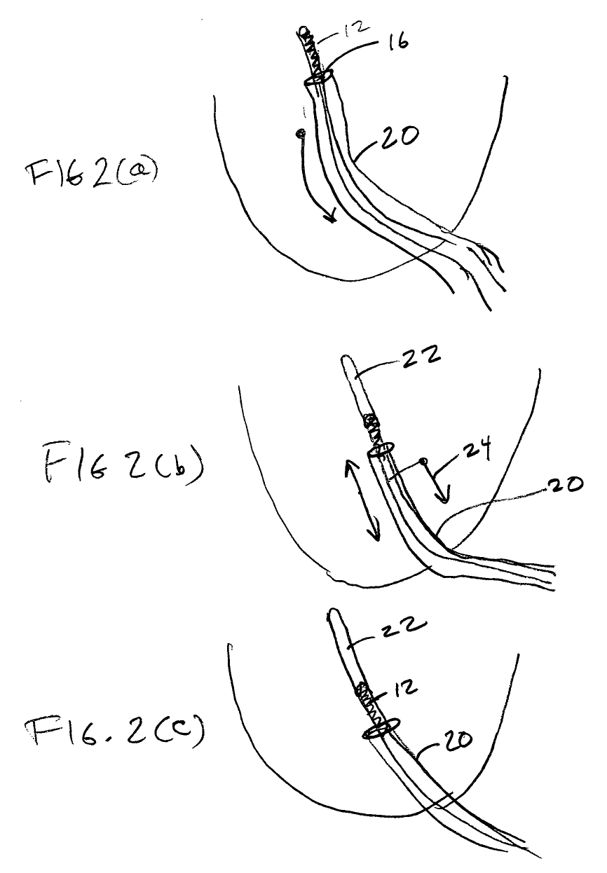

In FIG. 2a a ablation catheter is delivered tot he surface of the heart through the pericardial space. The ablation catheter 16 has an exposed electrode surface 12 which is used to deliver RF energy to the heart. The delivery sheath 20 is used to guide and stabilize the catheter on the cardiac surface. It is preferred to withdraw the electrode into the stationary delivery sheath 2 to create a “burn” or lesion. In Panel 2b the sheath 20 is moved as indicated by motion arrow 24 to a new position on the heart. Once again the electrode 22 is energized and dragged into he catheter to create a lesion shown by area 22. In panel 2c the process is repeated to lay down a linear lesion. In FIG. 1 and FIG. 2 the electrode surface 12 is the exposed coil of wire that makes up the catheter body.

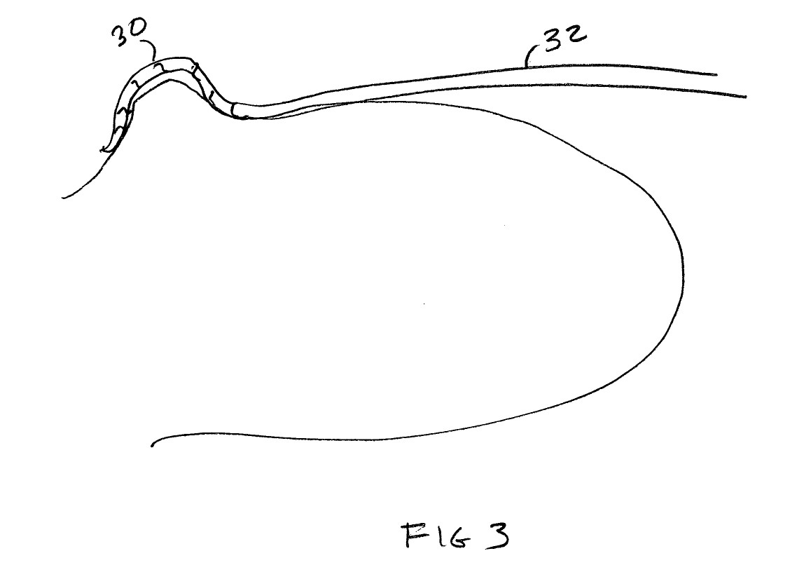

In FIG. 3 the electrode surface is made from a series of articulated links which are metallic and create a very flexible electrode surface. This allows the electrode 30 to conform to the complex surface of the heart. In this embodiment the catheter body 32 is of conventional coil construction but the distal tip is articulated.

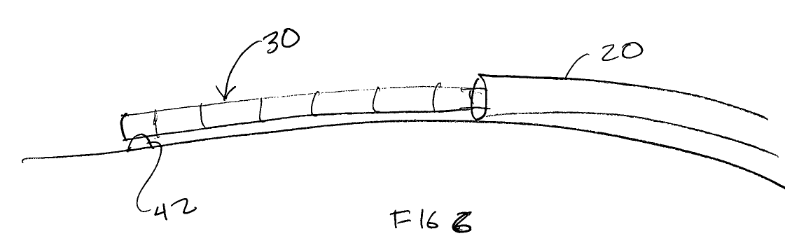

In FIG. 4 the center of the articulated distal tip is provided with a stylet or guide wire lumen 36 which allows a guide wire 38 to stiffen the structure during advancement and placement on the heart.

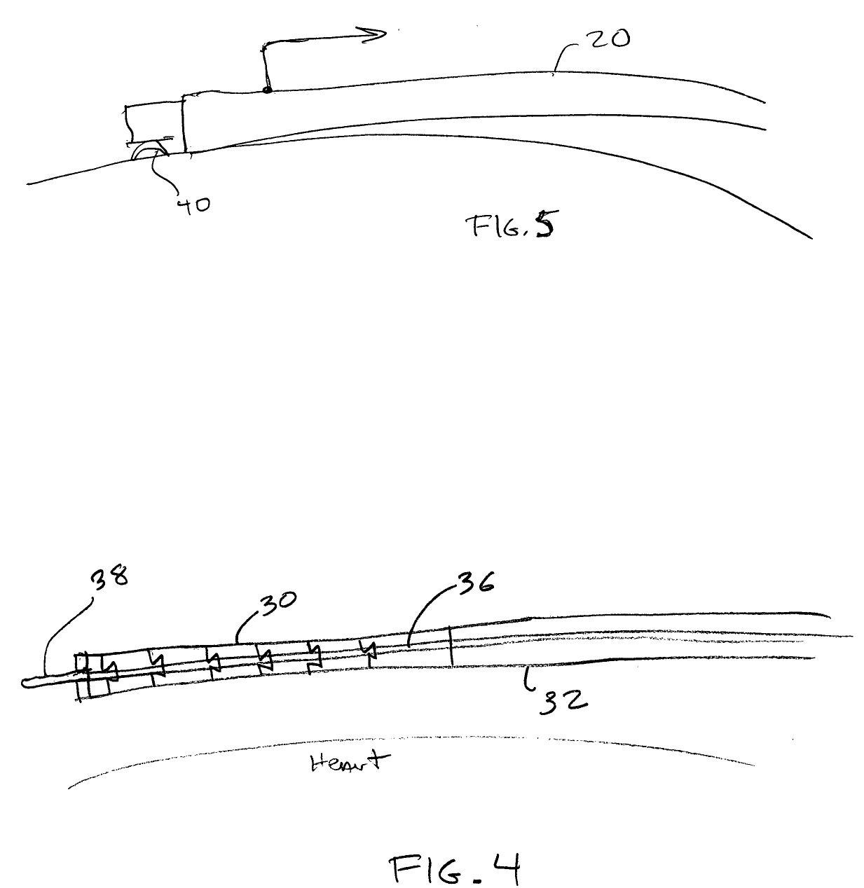

FIG. 5 shows an alternate anchor structure seen in the figure as a suction anchor 40. In FIG. 6 the anchor mechanism is a barbed hook. In either embodiment once the distal tip is anchored to the heart the sheath 20 may be retracted to permit a “burn”. One advantage is that the physician is certain that the lesion is “linear” and that all burn made from an anchored position are continuous.

What is claimed is:

1. A method of ablating the heart comprising the steps of:
a. fixing the tip of an RF catheter having a flexible body, to a location on the heart;
b. flexing the catheter body into conformity with the heart surface;
c. applying RF energy to said catheter.
